(19) United States
(12) Patent Application Publication
(10) Pub. No.: US 2004/0224943 A1
(43) Pub. Date: Nov. 11, 2004

(54) Method of lowering body temperature with (R) - 2,3-benzodiazepines

(76) Inventors: Steven M. Leventer, Langhorne, PA (US); Robert F. Kucharik, Glenmoore, PA (US)

Correspondence Address: BELL & ASSOCIATES, 416 FUNSTON ST., SUITE 100, SAN FRANCISCO, CA, 94118, US

(21) Appl. No.: 10/827,839

(22) Filed: Apr. 19, 2004

Related U.S. Application Data
(63) Continuation-in-part of application No. 10/781,422, filed on Feb. 17, 2004.
Application No. 10/781,422 is a continuation-in-part of application No. 10/369,823, filed on Feb. 19, 2003.

(51) Int. Cl. A61K 31/5513

(52) U.S. Cl. 514/221

(57) ABSTRACT

An (R)-2,3-benzodiazepine of Formula I, substantially isolated from the corresponding (S)-enantiomer thereof, is administered to lower the body temperature of an individual.

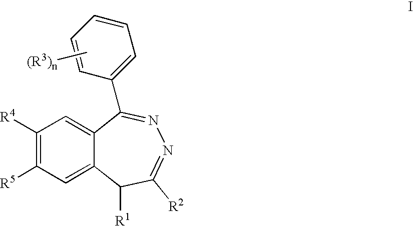

### CROSS-REFERENCE TO RELATED APPLICATIONS

This application is a continuation-in-part of copending U.S. application Ser. No. 10/781,422, filed Feb. 17, 2004, which is a continuation-in-part of copending U.S. application Ser. No. 10/369,823, filed Feb. 19, 2003, the entire disclosures of which are incorporated herein by reference.

### FIELD OF THE INVENTION

The present invention relates to methods of lowering body temperature, and particularly to methods of lowering increased body temperature associated with, for example, perimenopause or menopause.

### BACKGROUND OF THE INVENTION

2,3-Benzodiazepines-Tofisopam

Certain 2,3-benzodiazepines have been explored extensively for their potent CNS modulating activity. Compounds such as tofisopam (Grandaxin®), girisopam, and norisopam have demonstrated substantial anxiolytic and antipsychotic activity.

Tofisopam has been shown in humans to have an activity profile that is significantly different from that of widely used 1,4-benzodiazepine (BZ) anxiolytics such as diazepam (Valium®) and chlordiazepepoxide (Librium®). The 1,4-benzodiazepine, in addition to having sedative-hypnotic activity, also possess muscle relaxant and anticonvulsant properties which, though therapeutically useful in some disease states, are nonetheless potentially untoward side effects. Thus the 1,4-benzodiazepines, though safe when administered alone, may be dangerous in combination with other CNS drugs including alcohol.

Tofisopam, in contrast, is a non-sedative anxiolytic that has no appreciable sedative, muscle relaxant or anticonvulsant properties. See, Horvath et al., Progress in Neurobiology, 60 (2000), 309-342; the entire disclosure of which is incorporated herein by reference. In clinical studies, tofisopam improved rather than impaired psychomotor performance and showed no interaction with ethanol (Id.). These observations comport with data that show that tofisopam does not interact with central BZ receptors and binds only weakly to peripheral BZ receptors. Studies have also shown that tofisopam enhances mitogen-induced lymphocyte proliferation and IL-2 production in vitro.

Other 2,3-benzodiazepines that are structurally similar to tofisopam have been investigated and shown to have varying activity profiles. For example, GYKI-52466 and GYKI-53655 act as noncompetitive glutamate antagonists at the AMPA (α-amino-3-hydroxy-5-methyl-4-isoxazolepropionic acid) site and have demonstrated neuroprotective, muscle relaxant and anticonvulsant activity (Id.). Another group of 2,3-benzodiazepines that has been investigated are represented by the compound GYKI-52895, and show activity as selective dopamine uptake inhibitors with potential use in antidepressant and anti-Parkinsonism therapy.

Tofisopam is a racemic mixture of (R)—and (S)—enantiomers. This is due to the asymmetric carbon, i.e., a carbon with four different groups attached, at the 5-position of the benzodiazepine ring. The molecular structure and conformational properties of tofisopam have been determined by nuclear magnetic resonance (NMR), circular dichroism (CD), and X-ray crystallography. See, Visy et al., Chirality 1:271-275 (1989); the entire disclosure of which is incorporated herein by reference. The 2,3-diazepine ring exists as two different conformers. The major conformers, (+)R and (−)S have the 5-ethyl group in a quasi-equatorial position, while in the minor conformers, (−)R and (+)S, the 5-ethyl group is positioned quasi-axially. Thus, racemic tofisopam can exist as four molecular species, i.e., two enantiomers, each of which exists as two conformations. The sign of the optical rotation is reversed upon inversion of the diazepine ring from one conformer to the other. In crystal form, tofisopam exists only as the major conformations, with dextrorotatory tofisopam being of the (R) absolute configuration. See, Toth et al., J Heterocyclic Chem., 20:709-713 (1983); Fogassy et al., Bioorganic Heterocycles, Van der Plas, H. C., Otvos, L, Simongi, M., eds. Budapest Amsterdam: Akademia; Kiado-Elsevier, 229:233 (1984); the entire disclosures of which are incorporated herein by reference.

Differential binding of these two conformations of tofisopam has been reported in binding studies with human albumin. See, Simongi et al. Biochem. Pharm., 32(12), 1917-1920, 1983; the entire disclosure of which is incorporated herin by reference. The two conformers have also been reported as existing in equilibrium See, Zsila et al., Journal of Liquid Chromatography & Related Technologies, 22(5), 713-719, 1999; the entire disclosure of which is incorporated herein by reference.

The optically pure (R)-enantiomer of tofisopam (R)-1-(3,4-dimethoxyphenyl)-4-methyl-5-ethyl-7,8-dimethoxy-5H-2,3-benzodiazepine) has been isolated and shown to possess the nonsedative anxiolytic activity of the racemic mixture. See U.S. Pat. No. 6,080,736; the entire disclosure of which is incorporated herein by reference. The (S)-enantiomer of tofisopam (S)-1-(3,4-dimethoxyphenyl)-4-methyl-5-ethyl-7,8-dimethoxy-5H-2,3-benzodiazepine) has also been isolated and shown to possess an anticonvulsant activity and antiseizure activity not observed in the racemic mixture. See U.S. Pat. No. 6,649,607; the entire disclosure of which is incorporated herein by reference.

Metabolism of Tofisopam

Tofisopam has been shown to metabolize in human, rat, dog, monkey and rabbit to one or more of six major metabolites, depending on the host species:

| Compound # | Compound Name |
|---|---|
| 1 | 1-(3,4-dimethoxyphenyl)-4-methyl-5-ethyl-7-hydroxy-8- |
| | methoxy-5H-2,3-benzodiazepine |
| 2 | 1-(3,4-dimethoxyphenyl)-4-methyl-5-ethyl-7-methoxy-8- |
| | hydroxy-5H-2,3-benzodiazepine |
| 3 | 1-(3-methoxy-4-hydroxyphenyl)-4-methyl-5-ethyl-7,8- |
| | dimethoxy-5H-2,3-benzodiazepine |
| 4 | 1-(3-hydroxy-4-methoxyphenyl)-4-methyl-5-ethyl-7,8- |
| | dimethoxy-5H-2,3-benzodiazepine |
| 5 | 1-(3-methoxy-4-hydroxyphenyl)-4-methyl-5-ethyl-7- |
| | hydroxy-8-methoxy-5H-2,3-benzodiazepine |
| 6 | 1-(3-hydroxy-4-methoxyphenyl)-4-methyl-5-ethyl-7- |
| | hydroxy-8-methoxy-5H-2,3-benzodiazepine |

See Tomori et al., Journal of Chromatography, 241 (1982), p. 89-99.

Of the compounds named above, Compounds 1, 3, and 5 have been identified as metabolites in humans. These compounds have been synthesized and tested in certain pharmacological assays. See C. Ito, “Behavioral Pharmacological Study on the Structure Activity Relationship of Benzodiazepine Derivatives: With Particular Reference to the Activity of 2,3-Benzodiazepine,” J. Tokyo Med. College, 39:369-384 (1981).

In an assay of inhibition of aggression in mice, Compounds 1 and 3 showed 0% inhibition of aggression and Compound 5 showed a 28.6% inhibition of aggression. In an assay of muricide (mouse killing behavior) in rats, Compound 3 exhibited 0% inhibition of muricide while Compounds 1 and 5 each exhibited a 20% inhibition of muricide. In assays testing for anti-noradrenergic effects, Compound 1 exhibited no effect, while Compounds 3 and 5 demonstrated measurable activity. See Ito, Id.

Compounds 1, 3, 5, and 6 are also disclosed in U.S. Pat. No. 4,322,346, the entire disclosure of which is incorporated herein by reference. Compound 3 is reported therein to demonstrate narcosis-potentiating activity in mice. Furthermore, compounds 1 through 6 are disclosed for use in the treatment of irritable bowel syndrome and for nonulcer dyspepsia in U.S. Pat. No. 6,638,928, the entire disclosure of which is incorporated herein by reference.

Body Temperature-Fever

Body temperature in humans is controlled mostly by the hypothalamus. Regulation is achieved primarily from balance between heat loss from the periphery and heat production from tissues, particularly the liver and muscles. In health, the thermoregulatory center maintains body temperature of the internal organs from 37 to 38° C. (98.6° to 100.4° F.). Fever raises the hypothalamic set point, triggering the vasomotor center to begin vasoconstriction. Blood is then shunted from the periphery, decreasing the usual heat loss with a resultant increase in body temperature. Shivering, which increases heat production from muscle contraction, may also be triggered. Heat conservation and production continue until the temperature of the blood bathing the hypothalamic neurons reaches the new setting. The hypothalamus then maintains the new febrile temperature. Resetting the hypothalamic set point downward initiates the process of heat loss through sweating and vasodilation. See, The Merck Manual, Seventeenth Edition, p. 1093, 1999.

During a 24-hour period, temperature varies from lowest levels in the early morning to highest in late afternoon. The amplitude of this daily variation, the circadian temperature rhythm, is about 0.6° C. (1° F.). Id.

The cause of fever may be infectious or noninfectious (e.g., inflammatory, neoplastic, and immunologically mediated disorders). The pattern may be intermittent, characterized by daily spikes followed by a return to normal temperature, or remittent, in which the temperature does not return to normal. The elderly often have a diminished fever response. Certain patients, e.g., alcoholics, the very old, and the very young, may become hypothermic in response to severe infection. Id.

Pyrogens are substances that cause fever; they may be exogenous or endogenous. Exogenous pyrogens are derived from outside the host; most are microbes, microbial products, or toxins. The best studied are the lipopolysaccharides of gram-negative bacteria (commonly called endotoxin) and the toxin from Staphylococcus aureus strains isolated from patients with toxic shock syndrome. Id.

Exogenous pyrogens usually cause fever by inducing release of endogenous pyrogens (or so-called endogenous pyrogenic cytokines), which are polypeptides produced by various host cells, especially monocyte-macrophages. Other cells that produce fever-inducing cytokines include keratinocytes and endothelial, B, mesangial, epithelial, and glial cells. Endogenous pyrogens (interleukin-1, tumor necrosis factor, the interferons, and the gp 130 receptor-activating family [interleukin-6, interleukin-11, leukemia inhibitory factor, ciliary neurotropic factor, and oncostatin M]) cause fever by initiating metabolic changes in the hypothalamic thermoregulatory center. Prostaglandin E₂ synthesis appears to play a critical role. Id.

An ongoing debate exists over whether to treat a fever that occurs with an infectious disease. However, no clinical studies in humans support the benefit of fever (except, possibly, older studies of fever therapy for syphilis). In children at risk for seizures, fever should be treated. Antipyretic therapy also should be considered for febrile adults with preexisting cardiac or pulmonary insufficiency because fever can increase O₂ demands. For every 1° C. increase over 37° C. (99.5° F.), O₂ consumption increases 13%. Fever can also cause mental status changes in patients with dementia. Id.

Drugs that inhibit cyclooxygenase are effective in reducing fever; those used most often are acetaminophen, aspirin, and other non-steroidal anti-inflammatory drugs (NSAIDs). Although corticosteroids also reduce fever, they should not be used expressly for this purpose because of their other effects on the immune system.

Serotonin Syndrome

Serotonin syndrome is caused by excess stimulation of post-synaptic 5-hydroxytryptamine receptors in the brain stem and spinal cord, typically the result of combining serotonergic agents with monoamine oxidase inhibitors (MAOIs). There is no effective drug treatment established.

The symptoms of serotonin syndrome, mediated by the action of 5-hydroxytryptamine on various subtypes of serotonin receptors, include: euphoria, drowsiness, sustained rapid eye movement, overreaction of the reflexes, rapid muscle contraction and relaxation in the ankle causing abnormal movements of the foot, clumsiness, restlessness, feeling drunk and dizzy, muscle contraction and relaxation in the jaw, sweating, intoxication, muscle twitching, rigidity, high body temperature, shivering, diarrhea, loss of consciousness and death. See, The Serotonin Syndrome, Am. J. Psychiatry, June 1991; the entire disclosure of which is incorporated herein by reference.

Serotonin syndrome is generally caused by a combination of two or more drugs, one of which is often a selective sertonergic medication. The drugs which are known to frequently contribute to this condition are combinations of MAOIs with fluoetine (Prozac) and other selective serotonin reuptake inhibitors (SSRIs) or other drugs that have a powerful effect upon serotonin, i.e., clomipramine (Anafranil), trazadone (Deseryl), etc. The combination of lithium with these selective serotonergic agents has been implicated in enhancing serotonin syndrome. The tricyclic antidepressants, lithium, MAOIs, SSRIs, electric shock treatment, tryptophan, and the serotonin agonists (fenfluramine) all enhance serotonin neurotransmission and can contribute to the syndrome. Any factors that raise the level of serotonin can bring on this hyperserotonergic condition.

The published reports since 1982 indicate that in human patients, if the sertonergic medication is discontinued, the syndrome will often resolve on its own within twenty-four hours. Supportive measures can be used, however to ameliorate serious symptoms such as hyperthermia. These include cooling blankets for hyperthermia, intramuscular chlorpromazine as an antipyretic and sedative agent, artificial ventilation for respiratory insufficiency, anticonvulsants for seizures, clonazepam for myoclonus, and nifedipine for hypertension. See, A. B. Tracy, “Prozac: Panacea Or Pandora?” Cassia Publications, 1993, p. 88; the entire disclosure of which is incorporated herein by reference.

Malignant Hyperthermia

Malignant hyperthermia is a rare but potentially fatal metabolic syndrome. It is triggered in genetically predisposed patients by certain inhalational anesthetics, e.g., chloroform, ether, halothane, enflurane, isoflurane, sevoflurane, deflurane and depolarizing muscle relaxants, e.g., suxamethonium. Malignant hyperthermia manifests as a hypermetabolic state involving tachycardia, hypercarbia, base deficit, rigidity and fever. Many of the hallmark traits of an acute malignant hyperthermic crisis overlap with signs and symptoms of an emergent abdominal condition. Historically, there has been a reluctance in local community hospitals to manage patients known to be susceptible to malignant hyperthermia, and this is a source of frustration for many families in which there is a history of this condition. See, Heggie JE, Can. J. Surg. 2002 October; 45(5): 369-372; the entire disclosure of which is incorporated herein by reference.

Temperature Regulation Anomalies Resulting from Variation in Hormonal Levels

A. Temperature Regulation in Postmenopausal Women-Hot Flashes

The symptom of disturbance of normal thermoregulation, commonly referred to as “hot flashes” is a frequent clinical observation in postmenopausal women. The term “hot flash” refers to any sudden brief sensation of heat, often over the entire body, such as that experienced by many women during menopause. Hot flashes may also be drug induced by anti-estrogen compounds such as tamoxifen, toremifen, and raloxifen, or by removal of estrogen-producing tissues, e.g., abdominal hysterectomy and bilateral salpingo-oopherectomy. See, Loprinzi et al., Clin. Breast Cancer 2000 April; 1(1): 52-56; the entire disclosure of which is incorporated herein by reference. Drug induced hot flashes are not limited to women, occurring often in men undergoing cancer therapy, e.g., for example, tamoxifen therapy for prostate cancer.

Although hot flashes accompany the estrogen withdrawal coincident with menopause, estrogen alone is not responsible since estrogen levels do not differ significantly between patients who experience hot flashes and those who do not. See, Freedman, Am. J. Human Biol., 2001, Jul.-Aug.; 13(4): 453-464; the entire disclosure of which is incorporated herein by reference.

Estrogen replacement therapy is presently employed as a treatment for hot flashes. However this therapy is contraindicated in many patients, e.g., patients with breast cancer, personal history of breast cancer, or increased risk of breast cancer; patients with a thromboembolic disease; patients with coronary artery disease; patients with undiagnosed vaginal bleeding; patients with migraine; and patients with seizure disorders. See, Menopause: The Journal of the North American Menopause Society, Vol. 7, No. 2, pp. 76-86; the entire disclosure of which is incorporated herein by reference.

B. Hot Flashes Other than Postmenopausal

The time interval which represents the transition from normal menstrual function to menopause has been termed “perimenopause.” This interval can extend up to about ten years prior to the complete cessation of menstrual cycles. The phenomenon of hot flashes is common throughout the transition interval of perimenopause, often occurring prior to any other symptomatic indicia of approaching menopause.

C. Agents Useful in Treatment of Menopausal Symptoms

Numerous chemical entities have been investigated for biological activity in the symptomatic treatment of menopause. Particular classes of compounds which have been investigated include estrogen agonists, progesterone agonists, drug formulations comprising both an estrogen agonist and a progesterone agonist, selective estrogen receptor modulators, bisphosphonates, selective serotonin reuptake inhibitors (SSRIs), norepinephrine serotonin reuptake inhibitors (NSRIs), and gamma aminobuteric acid (GABA) modulators.

Exemplary compounds of interest that have been shown to possess activity in treating menopause are listed in Table 1.

TABLE 1

| Drug Class | Exemplary compounds |
|---|---|
| estrogen agonists | estradiol |
| Formulations comprising an estrogen | estradiol/trimegestrone |
| agonist and a progesterone agonist | |
| progesterone agonists | trimegestrone |
| selective estrogen receptor modulators | raloxifene |
| | bazedoxifene |
| bisphosphonates | risedronic acid |
| | ibandronic |
| SSRIs | fluoxetine |
| | paroxetine |
| NSRI | venlafaxine |
| GABA modulator | gabapentin |

Stroke

Lowering body temperature may improve an individual's chances for long-term survival after a stroke.

In a study of 390 patients who were admitted within six hours (median 2.4 hours) of suffering a stroke, researchers found that in acute human stroke, an association exists between body temperature and initial stroke severity, infarct size, mortality, and outcome.

In a prospective and consecutive study 390 stroke patients were admitted to hospital within 6 hours after stroke. For the study, there was a determination of body temperature on admission, initial stroke severity, infarct size, mortality, and outcome in survivors. Stroke severity was measured on admission, weekly, and at discharge on the Scandinavian Stroke Scale (SSS). Infarct size was determined by computed tomography. Multiple logistic and linear regression outcome analyses included relevant confounders and potential predictors such as age, gender, stroke severity on admission, body temperature, infections, leucocytosis, diabetes, hypertension, atrial fibrillation, ischemic heart disease, smoking previous stroke, and comorbidity.

The study found that mortality was lower and outcome better in patients with mild hypothermia on admission; both were worse in patients with hyperthermia. Body temperature was independently related to initial stroke severity (p<0.009), infarct size (p<0.0001), mortality (p<0.02), and outcome in survivors (SSS at discharge) (p<0.003). For each 1 degree Celsius increase in body temperature the relative risk of poor outcome (death or SSS score on discharge <30 points) rose by 2.2 (95% CL 1.4-3.5) (p<0.002). Patients with a body temperature of more than 37 degrees Celsius had more severe strokes, and also had a higher mortality rate five years after their strokes occurred. See, Jorgensen et al., Lancet, 1996, Feb. 17; 347(8999): 422-425.

Elevated body temperature increases mortality and worsens outcome in acute stroke patients. In animal models of stroke, even slight hypothermia was shown to be neuroprotective. Pharmacological treatment alone (paracetamol, metamizol) usually fails to lower core body temperature below 37 degrees C. See, Knoll et al., J. Neurosurg. Anesthesiol., 2002, Oct.; 14(4): 304-308.

What is needed are new agents that effectively lower body temperature in instances wherein the body temperature is abnormally high and in instances wherein lowering the body temperature to a level below normal body temperature provides a therapeutic benefit.

### SUMMARY OF THE INVENTION

According to one embodiment of the invention there is provided a method of lowering body temperature of an individual which is a mammal, particularly a human, comprising administering to the individual an effective amount of at least one compound according to Formula I:

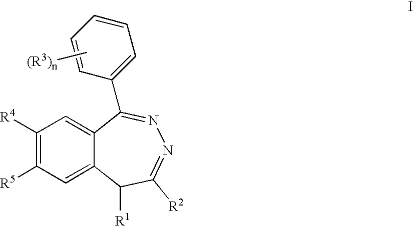

wherein:

R¹ is —(C₁-C₇)hydrocarbyl, preferably —(C₁-C₆)alkyl, more preferably —(C₁-C₃)alkyl, most preferably methyl or ethyl, or —(C₂-C₆)heteroalkyl;

R² is selected from the group consisting of —H, and —(C₁-C₇)hydro-carbyl, preferably —(C₁-C₆)alkyl, more preferably —(C₁-C₃)alkyl, most preferably methyl and ethyl, wherein R¹ and R² may combine to form a carbocyclic or heterocyclic 5- or 6-membered ring;

R^{3a}, R^{3b} and R^{3c} are independently selected from the group consisting of —H; —O(C₁-C₇)hydrocarbyl, preferably —O(C₁-C₆)alkyl, more preferably —O(C₁-C₃)alkyl, most preferably methoxy and ethoxy; —OH; —OC(═O)(C₁-C₆)alkyl, preferably —OC(═O)(C₁-C₆)alkyl, more preferably —OC(═O)CH₃ and —OC(═O)CH₂CH₃; —OC(═O)O(C₁-C₇)hydrocarbyl, preferably —OC(═O)O(C₁ —C₆)alkyl and —OC(═O)O-benzyl, more preferably —OC(═O)OCH₃, —OC(═O)OCH₂CH₃, and —OC(═O)O-benzyl; —SH; —S(C₁-C₃)alkyl, preferably —SCH₃; —NH₂; —NH(C₁-C₆)alkyl, preferably —NH(C₁-C₃)alkyl, more preferably —NHCH₃ and —NHCH₂CH₃; —N((C₁-C₆)alkyl)₂, preferably —N((C₁-C₃)alkyl)₂, more preferably —N(CH₃)₂, —N(CH₂CH₃)₂ and —N(CH₃)CH₂CH₃; —NH(═O)(C₁-C₆)alkyl, preferably —NHC(═O)(C₁-C₃)alkyl, more preferably —NHC(═O)CH₃ and —NHC(═O)CH₂CH₃; —NO₂; and halogen;

provided at least one of R^{3a}, R^{3b} and R^{3c} is other than —H;

R⁴ and R⁵ are independently selected from the group consisting of —O(C₁-C₇)hydrocarbyl, preferably —O(C₁-C₆)alkyl, more preferably —O(C₁-C₃)alkyl, most preferably methoxy and ethoxy; —OH; —OC(═O)(C₁-C₆)alkyl, preferably —OC(═O)(C₁-C₆)alkyl, more preferably —OC(═O)CH₃ and —OC(═O)CH₂CH₃; —OC(═O)O(C₁-C₇)hydrocarbyl, preferably —OC(═O)O(C₁ —C₆)alkyl and —OC(═O)O-benzyl, more preferably —OC(═O)OCH₃, —OC(═O)OCH₂CH₃, and —OC(═O)O-benzyl; —SH; —S(C₁-C₃)alkyl, preferably —SCH₃; —NH₂; —NH(C₁-C₆)alkyl, preferably —NH(C₁-C₃)alkyl, more preferably —NHCH₃ and —NHCH₂CH₃; —N((C₁-C₆)alkyl)₂, preferably —N((C₁-C₃)alkyl)₂, more preferably —N(CH₃)₂, —N(CH₂CH₃)₂ and —N(CH₃)CH₂CH₃; —NH(═O)(C₁-C₆)alkyl, preferably —NHC(═O)(C₁-C₃)alkyl, more preferably —NHC(═O)CH₃ and —NHC(═O)CH₂CH₃; —NO₂; and halogen;

wherein R⁴ and R⁵ may combine to form a 5-, 6- or 7-membered heterocyclic ring, preferably a 5- or 6-membered heterocyclic ring, more preferably a 5-membered heterocyclic ring; and

wherein the at least one administered compound according to Formula I comprises an (R)-enantiomer, substantially free of the corresponding (S)-enantiomer, with respect to the absolute conformation at the 5-position of the benzodiazepine ring; or

a pharmaceutically-acceptable salt of such a compound.

Preferably, R⁴ and R⁵ are independently selected from the group consisting of —O(C₁-C₆)alkyl, —OC(═O)(C 1-C₆)alkyl, —OC(═O)O(C₁-C₇)hydrocarbyl and —OH; and

R^{3a}, R^{3b} and R^{3c} are independently selected from the group consisting of —H, —O(C₁-C₆)alkyl, —OC(═O)(C₁-C₆)alkyl, —OC(═O)O(C₁-C₇)hydrocarbyl and —OH;

wherein at least one of R^{3a}, R^{3b} and R^{3c} is other than —H.

More preferably, R⁴ and R⁵ are independently selected from the group consisting of —OH and 4(C₁-C₆)alkyl; and

R^{3a}, R^{3b} and R^{3c} are independently selected from the group consisting of —H, —OH and —O(C₁-C₆)alkyl.

Most preferably, R⁴ and R⁵ are independently selected from the group consisting of —OH and —OCH₃.

Preferably, when one or two of R^{3a}, R^{3b} and R^{3c} are other than —H, they will be at the 2- or 3-position, or both the 2- and 3-positions, of the phenyl ring to which they are attached.

According to a preferred sub-embodiment, the compounds according to Formula I for administration are compounds according to Formula 11:

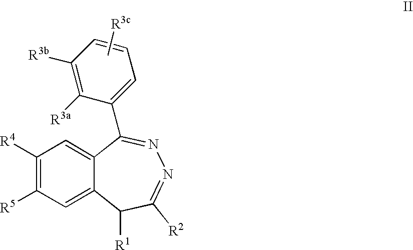

wherein:

R¹ is —(C₁-C₇)hydrocarbyl, preferably —(C₁-C₆)alkyl, more preferably —(C₁-C₃)alkyl, most preferably methyl or ethyl, or —(C₂-C₆)heteroalkyl;

R² is selected from the group consisting of —H, and —(C₁-C₇)hydro-carbyl, preferably —(C₁-C₆)alkyl, more preferably —(C₁-C₃)alkyl, most preferably methyl and ethyl, wherein R¹ and R² may combine to form a carbocyclic or heterocyclic 5- or 6-membered ring;

R^{3a}, R^{3b} and R^{3c} are independently selected from the group consisting of —H; —O(C₁-C₇)hydrocarbyl, preferably —O(C₁-C₆)alkyl, more preferably 4)(C₁-C₃)alkyl, most preferably methoxy and ethoxy; —OH; —OC(═O)(C₁-C₆)alkyl, preferably —OC(═O)(C₁-C₆)alkyl, more preferably —OC(═O)CH₃ and —OC(═O)CH₂CH₃; —OC(═O)O(C₁-C₇)hydrocarbyl, preferably —OC(═O)O(Cl —C₆)alkyl and —OC(═O)O-benzyl, more preferably —OC(═O)OCH₃, OC(═O)OCH₂C H 3, and —OC(═O)O-benzyl; —SH; —S(C₁-C₃)alkyl, preferably —SCH₃; —NH₂; —NH(C₁-C₆)alkyl, preferably —NH(C₁-C₃)alkyl, more preferably —NHCH₃ and —NHCH₂CH₃; —N((C₁-C₆)alkyl)₂, preferably —N((C₁-C₃)alkyl)₂, more preferably —N(CH₃)₂, —N(CH₂CH₃)₂ and —N(CH₃)CH₂CH₃; —NH(═O)(C₁-C₆)alkyl, preferably —NHC(═O)(C₁-C₃)alkyl, more preferably —NHC(═O)CH₃ and —NHC(═O)CH₂CH₃; —NO₂; and halogen;

provided at least one of R^{3a} and R^{3b} is other than —H;

R⁴ and R⁵ are independently selected from the group consisting of —O(C₁-C₇)hydrocarbyl, preferably —O(C₁-C₆)alkyl, more preferably —O(C₁-C₃)alkyl, most preferably methoxy and ethoxy; —OH; —OC(═O)(C₁-C₆)alkyl, preferably —OC(═O)(C₁-C₆)alkyl, more preferably —OC(═O)CH₃ and —OC(═O)CH₂CH₃; —OC(═O)O(C₁-C₇)hydrocarbyl, preferably —OC(═O)O(C₁ —C₆)alkyl and —OC(═O)O-benzyl, more preferably —OC(═O)OCH₃, —OC(═O)OCH₂C H 3, and —OC(═O)O-benzyl; —SH; —S(C₁-C₃)alkyl, preferably —SCH₃; —NH₂; —NH(C₁-C₆)alkyl, preferably —NH(C₁-C₃)alkyl, more preferably —NHCH₃ and —NHCH₂CH₃; —N((C₁-C₆)alkyl)₂, preferably —N((C₁-C₃)alkyl)₂, more preferably —N(CH₃)₂, —N(CH₂CH₃)₂ and —N(CH₃)CH₂CH₃; —NH(═O)(C₁-C₆)alkyl, preferably —NHC(═O)(C₁-C₃)alkyl, more preferably —NHC(═O)CH₃ and —NHC(═O)CH₂CH₃; —NO₂; and halogen;

wherein R⁴ and R⁵ may combine to form a 5-, 6- or 7-membered heterocyclic ring, preferably a 5- or 6-membered heterocyclic ring, more preferably a 5-membered heterocyclic ring; and

wherein the at least one administered compound according to Formula I comprises an (R)-enantiomer, substantially free of the corresponding (S)-enantiomer, with respect to the absolute conformation at the 5-position of the benzodiazepine ring; or

a pharmaceutically-acceptable salt of such a compound.

I. First Embodiment of the Formula II Compounds

According to a First Embodiment of the compounds according to Formula II for administration:

R¹ and R² are defined as for Formula II;

R^{3c} is —H;

one or two of R^{3a}, R^{3b}, R⁴, and R¹ is —OH; and

the remaining members of the group R^{3a}, R^{3b}, R⁴, and R¹ are independently selected from the group consisting of —O(C₁-C₇)hydrocarbyl, preferably —O(C₁-C₆)alkyl, more preferably —O(C₁-C₃)alkyl, most preferably methoxy or ethoxy; —OC(═O)(C₁-C₆)alkyl, preferably —OC(═O)(C₁-C₆)alkyl, more preferably —OC(═O)CH₃ and —OC(═O)CH₂CH₃; —OC(═O)O(C₁-C₇)hydrocarbyl, preferably —OC(═O)O(C₁-C₆)alkyl and —OC(═O)O-benzyl, more preferably —OC(═O)OCH₃, —OC(═O)OCH₂CH₃, and —OC(═O)O-benzyl; —OH; —SH; —S(C₁-C₃)alkyl, preferably —SCH₃; —NH₂; —NH(C₁-C₆)alkyl, preferably —NH(C₁-C₃)alkyl, more preferably —NHCH₃ and —NHCH₂CH₃; —N((C₁-C₆)alkyl)₂, preferably —N((C₁-C₃)alkyl)₂, more preferably —N(CH₃)₂, —N(CH₂CH₃)₂ and —N(CH₃) CH₂CH₃; —NH(═O)(C₁-C₆)alkyl, preferably —NHC(═O)(C₁-C₃)alkyl, more preferably —NHC(═O)CH₃ and —NHC(═O)CH₂CH₃; —NO₂; and halogen;

wherein R⁴ and R⁵ may combine to form a 5-, 6- or 7-membered heterocyclic ring, preferably a 5- or 6-membered heterocyclic ring, more preferably a 5-membered heterocyclic ring.

According to a Sub-embodiment of compounds of Formula II;

one or two of R^{3a}, R^{3b}, R⁴, and R⁵ is —OH;

one of the remaining members of the group R^{3a}, R^{3b}, R⁴, and R⁵ is —O(C₁-C₇)hydrocarbyl, preferably —O(C₁-C₆)alkyl, more preferably —O(C₁ —C₃)alkyl, most preferably methoxy or ethoxy; and

the remaining members of the group R^{3a}, R^{3b}, R⁴, and R⁵ are independently selected from the group consisting of —O(C₁-C₇)hydrocarbyl, —OC(═O)(C₁ —C₆)alkyl, —SH, —S(C₁-C₃)alkyl, —NH₂, —NH(C₁-C₆)alkyl, —N((C₁-C₆)alkyl)₂, —NH(═O)(C₁-C₆)alkyl, —NO2; and halogen;

wherein R⁴ and R⁵ may combine to form a 5-, 6- or 7-membered heterocyclic ring, preferably a 5- or 6-membered heterocyclic ring, more preferably a 5-membered heterocyclic ring.

According to a preferred group within said Sub-embodiment of compounds of Formula II;

one or two of R^{3a}, R^{3b}, R⁴, and R¹ is —OH; and

the remaining members of the group R^{3a}, R^{3b}, R⁴, R⁵ are independently selected from the group consisting of —O(C₁-C₇)hydrocarbyl, preferably —O(C₁-C₆)alkyl, more preferably —O(C₁-C₃)alkyl, most preferably methoxy or ethoxy.

According to some sub-embodiments within said preferred group, R^{3a} or R^{3b} is —OH.

According to other sub-embodiments within said preferred group, R⁴ is —OH.

According to still other sub-embodiments within said preferred group, R⁵ is —OH.

Preferred compounds according the First Embodiment of compounds according to Formula II are selected from the group consisting of:

(R)-1-(3,4-dimethoxyphenyl)-4-methyl-5-ethyl-7-hydroxy-8-methoxy-5H-2,3-benzodiazepine;

(R)-1-(3-hydroxy-4-methoxyphenyl)-4-methyl-5-ethyl-7,8-dimethoxy-5H-2,3-benzodiazepine;

(R)-1-(3-methoxy-4-hydroxyphenyl)-4-methyl-5-ethyl-7,8-dimethoxy-5H-2,3-benzodiazepine;

(R)-1-(3,4-dimethoxyphenyl)-4-methyl-5-ethyl-7-methoxy-8-hydroxy-5H-2,3-benzodiazepine;

(R)-1-(3-methoxy-4-hydroxyphenyl)-4-methyl-5-ethyl-7-hydroxy-8-methoxy-5H-2,3-benzodiazepine;

(R)-1-(3-hydroxy-4-methoxyphenyl)-4-methyl-5-ethyl-7-hydroxy-8-methoxy-5H-2,3-benzodiazepine; and

pharmaceutically-acceptable salts of such compounds.

II. Second Embodiment of the Formula II Compounds

According to a Second Embodiment of the compounds of Formula II for administration; R¹ and R are defined as for Formula II;

R^{3c} is —H; and

R^{3a}, R^{3b}, R⁴, and R⁵ are independently selected from the group consisting of —O(C₁-C₇)hydrocarbyl, preferably —O(C₁-C₆)alkyl, more preferably —O(C₁-C₃)alkyl, most preferably methoxy or ethoxy.

Most preferably, the compound according to the Second Embodiment of a compound of Formula II is (R)-tofisopam, substantially isolated from the corresponding (S)-enantiomer of tofisopam, or a pharmaceutically-acceptable salt thereof.

Preferably, the (R)-enantiomer of the compound administered according to the present invention is 85% or more by weight of the total weight of the compound administered. More preferably, the (R)-enantiomer is 90% or more by weight of the total weight of the compound. Still more preferably, the (R)-enantiome is 95% or more by weight of the total weight of the compound. Most preferably, the (R)-enantiomer of the compound administered according to the present invention is 99% or more by weight of the total weight of the compound.

According to another embodiment of the invention there is provided a method of lowering the body temperature of an individual suffering from hot flashes, particularly, hot flashes associated with menopause, said method comprising administering to the individual an effective amount of at least one compound according to Formula I as defined herein, and at least one additional therapeutic agent.

Preferably, the at least one additional therapeutic agent is selected from the group consisting of estrogen agonists, progesterone agonists, selective estrogen receptor modulators, bisphosphonates, selective serotonin reuptake inhibitors (SSRIs), norepinephrine serotonin reuptake inhibitors (NSRIs) and gamma aminobuteric acid (GABA) modulators.

According to yet another embodiment of the invention, there is provided a composition comprising at least one compound of Formula I as defined herein, and at least one additional therapeutic agent, wherein the at least one additional therapeutic agent is selected from the group consisting of estrogen agonists, progesterone agonists, selective estrogen receptor modulators, bisphosphonates, selective serotonin reuptake inhibitors (SSRIs), norepinephrine serotonin reuptake inhibitors (NSRIs) and gamma aminobuteric acid (GABA) modulators.

### Definitions

The phrase “optically active” refers to a property whereby a material rotates the plane of plane-polarized light. A compound that is optically active is nonsuperimposable on its mirror image. The property of nonsuperimposablity of an object on its mirror image is called chirality.

The property of “chirality” in a molecule may arise from any structural feature that makes the molecule nonsuperimposable on its mirror image. The most common structural feature producing chirality is an asymmetric carbon atom, i.e., a carbon atom having four nonequivalent groups attached thereto.

The term “enantiomer” refers to each of the two nonsuperimposable isomers of a pure compound that is optically active. Single enantiomers are designated according to the Cahn-Ingold-Prelog system, a set of priority rules that rank the four groups attached to an asymmetric carbon. See March, Advanced Organic Chemistry, 4^{th} Ed., (1992), p. 109. Once the priority ranking of the four groups is determined, the molecule is oriented so that the lowest ranking group is pointed away from the viewer. Then, if the descending rank order of the other groups proceeds clockwise, the molecule is designated R and if the descending rank of the other groups proceeds counterclockwise, the molecule is designated S. In the example below, the Cahn-Ingold-Prelog ranking sequence id A>B>C>D. The lowest ranking atom, D is oriented away from the viewer.

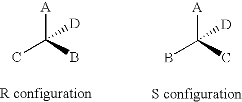

The term “racemate” or the phrase “racemic mixture” refers to a 50-50 mixture of two enantiomers of a compound such that the mixture does not rotate plane-polarized light.).

The term “substantially isolated”, or “substantially free of the other enantiomer” or the term “resolved” when used to refer to an optically active compound, means the (R)- and (S)-enantiomers of the compound have been separated such that the composition is 80% or more by weight a single enantiomer.

Likewise, the expression, “an (R)-enantiomer, substantially free of the corresponding (S)-enantiomer” refers herein to a compound of Formula I that comprises 80% or more by weight of the (R)-enantiomer and likewise contains 20% or less of the (S)-enantiomer as a contaminant, by weight. The “corresponding (S)-enantiomer” refers to the compound that is the (S)-enantiomer which is the optical isomer of the specific (R)-enantiomer that comprises the active agent of the compound of Formula I. Thus, by the expression “(R)-tofisopam substantially free of the (S)-enantiomer” is meant tofisopam that comprises 80% or more by weight of the (R)-enantiomer and likewise contains 20% or less of the (S)-enantiomer as a contaminant, by weight.

The term “effective amount” when used to describe therapy to a patient to lower body temperature, refers to the amount of a compound of Formula I that results in a therapeutically useful reduction in body temperature when administered to a patient suffering from a disorder which manifests elevated body temperature. Further, the term “effective amount” may be used to refer to the amount of a compound of Formula I that results in a therapeutically useful reduction in body temperature when administered to a patient suffering from disorder which is effectively treated by lowering body temperature.

The term “effective amount” when used to describe therapy to lower the body temperature of an individual suffering from hot flashes, particularly hot flashes associated with menopause, refers to the amount of a compound of Formula I, or of a combination of a compound of Formula I with one or more additional agents, e.g., estrogen agonists, progesterone agonists, selective estrogen receptor modulators, bisphosphonates, SSRIs, NSRIs and (GABA) modulators.

The term “individual” or “subject”, includes human beings and non-human animals.

The term “alkyl”, by itself or as part of another substituent means, unless otherwise stated, a straight, branched or cyclic chain hydrocarbon radical, including di- and multi-radicals, having the number of carbon atoms designated (i.e. C₁-C₆ means one to six carbons) and includes straight, branched chain or cyclic groups. Examples include: methyl, ethyl, propyl, isopropyl, butyl, isobutyl, tert-butyl, pentyl, neopentyl, hexyl, cyclohexyl and cyclopropylmethyl. Most preferred is (C₁-C₃)alkyl, particularly ethyl, methyl and isopropyl.

The term “hydrocarbyl” refers to any moiety comprising only hydrogen and carbon atoms. Preferred is (C₁-C₇)hydrocarbyl, more preferably (C₁-C₆)alkyl and benzyl.

The term “heteroalkyl” by itself or in combination with another term means, unless otherwise stated, a stable straight or branched chain radical consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂-CH₂-CH₃, —CH₂-CH₂CH₂—OH, —CH₂-CH₂—NH—CH₃, —CH₂—S—CH₂-CH₃, and —CH₂ CH₂—S(═O)—CH₃. Up to two heteroatoms may be consecutive, such as, for example, —CH₂—NH—OCH₃.

The term “heterocycle” or “heterocyclyl” or “heterocyclic” by itself or as part of another substituent means, unless otherwise stated, an unsubstituted or substituted, stable, mono- or multicyclic heterocyclic ring system which consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

The term “heteroaryl” or “heteroaromatic” refers to a heterocycle having aromatic character.

Examples of non-aromatic heterocycles include monocyclic groups such as: pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 1,4-dioxepane, 4,7-dihydro-1,3-dioxepin and hexamethyleneoxide.

Examples of heteroaryl groups include: pyridine, pyrazine, pyrimidine, pyridazine, thiophene, furan, pyrroline, imidazoline, thiazoline, oxazoline, pyrazoline, isothiazoline, 1,2,3-triazoline, 1,2,3-thiadiazoline, 1,2,3-oxadiazoline.

Examples of polycyclic heterocycles include: Indole, indoline, quinoline, tetrahydroquinoline, isoquinoline, tetrahydroisoquinoline, cinnoline, quinoxaline, quinazoline, 1,4-benzodioxolane, 1,4-benzodioxepane, 1,3-benzodioxane, and coumarin, dihydrocoumarin, benzofuran, 2,3-dihydrobenzofuran, 1,2-benzisoxazoline, benzothiophene, benzoxazoline, benzthiazoline, purine, benzimidazoline, particularly 2-benzimidazoline, benztriazoline, thioxanthine, carbazole, carboline, acridine, pyrrolizidine, and quinolizidine.

The aforementioned listing of heterocyclyl and heteroaryl moieties is intended to be representative, not limiting.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position.

### DESCRIPTION OF THE FIGURES

FIG. 1 is a plot of body temperature data gathered in the Stress Induced Hyperthermia (SIH) assay, comparing the body temperature lowering effect of chlordiazepoxide (CDP), (R)-tofisopam, (S)-tofisopam and racemic tofisopam (tofisopam R+S).

FIG. 2 is a bar graph showing the measured core body temperature of test animals at Ti of the SIH assay, comparing the body temperature lowering effect of chlordiazepoxide (CDP), (R)-tofisopam, (S)-tofisopam and racemic tofisopam (tofisopam R+S).

### DETAILED DESCRIPTION OF THE INVENTION

According to the present invention, (R)-2,3-benzodiazepines of Formula I,

and pharmaceutically acceptable salts thereof, are useful in methods for lowering body temperature.

(R)-tofisopam has demonstrated therapeutic activity in the Stress-Induced Hypothermia (SIH) Model, an animal model designed to demonstrate the activity of pharmacological hypothermic agents. In this assay, two successive temperature measurements are performed, the first, a basal measurement and the second, a measurement of a stress-enhanced temperature. The difference between the two measurements (delta T) is compared in animals treated with a test compound versus animals treated with vehicle alone to determine the test compound's activity in lowering body temperature. Anxiolytics such as classical 1,4-benzodiazepines and 5-HT_{1A} receptor agonists reduce delta T, whereas antidepressants do not reduce delta T. See, Olivier, et al., “Anxiolytic effects of flesinoxan in the stress-induced hyperthermia paradigm in singly-housed mice are 5-HT_{1A} receptor mediated,” European Journal of Pharmacology 342:177-182, (1998). Besides the effect of drugs on the stress-enhanced temperature (T₂), this test also directly measures intrinsic effects of drugs on the core body temperature (T₁). Thus a test compound that reduces T₂ and thus yields a lower delta T has utility in therapies that are directed to lowering the body temperature of an individual, such as perimenopause and menopause. Furthermore, the tolerability of (R)-tofisopam is greater than that of other compounds tested by us, including (S)-tofisopam and the racemate.

Body temperature and emotional states are also closely related in humans. See, Marazziti et al., Psychological stress and body temperature changes in humans, Physiology and Behavior, (1992), 52:393-395; and Reeves et al., “Endogenous hyperthermia in normal human subjects: Experimental study of emotional states (II),” International Journal of Psychosomatics, (1985), 32:18-23. Changes in autonomic functioning are routinely considered when diagnosing generalized anxiety disorder. Thus, stress-induced hyperthermia in mice is also considered to have good predictive, and construct validity for certain anxiety/stress disorders in humans, with relatively few false positives/false negatives. See, Zethof et al., “Stress induced hyperthermia as a putative anxiety model,” European Journal of Pharmacology, (1995), 294:125-135.

According to one embodiment of the invention, there is provided a method of lowering the body temperature of an individual afflicted with a disorder associated with an elevated body temperature, said method comprising administering to the individual an effective amount of at least one compound of Formula I.

Such disorders include, but are not limited to, fever, malignant hyperthermia, and serotonin syndrome.

Substances that are capable of lowering body temperature are useful in the treatment of hot flashes. See, for example, U.S. Pat. No. 6,310,098. Thus, disorders associated with an elevated body temperature, treatable according to the present invention, include hot flashes. Hot flashes are treatable by administration of at least one compound of Formula I, or by administration of at least one compound of Formula I in combination with at least one additional therapeutic agent.

Hot flashes treatable by the method of the invention include, for example, hot flashes associated with variation in hormone levels, e.g., those occurring during menopause or perimenopause; hot flashes which occur as a side-effect of a drug therapy, for example an anti-estrogen therapy comprising administration of tamoxifen, toremifen or raloxifen, for example; and hot flashes that occur subsequent to the removal of estrogen-producing tissues, e.g., ovarectomy, abdominal hysterectomy and bilateral salpingo-oopherectomy.

In another embodiment of the invention there is provided a method of lowering the body temperature of an individual afflicted with a disorder wherein therapeutic benefit results from lowering of the body temperature to a level below the normal body temperature, said method comprising administering to the individual an effective amount of at least one compound of Formula I as described above.

Such disorders include stroke and cerebral ischemia. Thus, a method for treating or preventing the neuronal damage associated with cerebral ischemia is provided, comprising administering to an individual in need of such treatment an effective amount of at least one compound of Formula I.

The compounds of Formula I useful in the present invention may be prepared by one of several methods. These methods generally begin with synthetic strategies and procedures used in the synthesis of racemic 2,3-benzodiazepines, e.g., for example, tofisopam and further include a resolution of the racemate to isolate the (R)-enantiomer, substantially free of the corresponding (S)-enantiomer. See U.S. Pat. Nos. 3,736,315 and 4,423,044 (tofisopam syntheses) and Horvath et al., Progress in Neurobiology 60(2000) p. 309-342 and references cited therein (preparation of tofisopam and analogs thereof), the entire disclosures of which are incorporated herein by reference.

In the synthesis methods that follow, the product of the chemical syntheses is a racemic 2,3-benzodiazepine. This racemic mixture is subsequently separated using known methods of resolution to produce the (R)-2,3-benzodiazepine of Formula I, substantially free of the corresponding (S)-enantiomer. The synthesis methods are shown herein for tofisopam as exemplary of synthesis of racemates containing the (R)-enantiomer compounds of Formula I.

Preferably, the compound used in methods of the present invention has a composition that is 85% by weight or greater of the (R)-2,3-benzodiazepine of Formula I, and 15% by weight, or less, of the (S)-enantiomer. More preferably, the compound used in methods of the present invention has a composition that is 90% by weight or greater of (R)-2,3-benzodiazepine of Formula 1 and 10% by weight, or less, of the (S)-enantiomer. Still more preferably, the compound used in methods of the present invention has a composition that is 95% by weight or greater of (R)-2,3-benzodiazepine of Formula 1 and 5% by weight, or less, of the corresponding (S)-enantiomer. Most preferably, the compound used in methods of the present invention has a composition that is 99% by weight or greater of (R)-2,3-benzodiazepine of Formula 1 and 1% by weight, or less, of the corresponding (S)-enantiomer.

Racemic mixtures containing (R)-enantiomer compounds of Formula 1 may be synthesized, as shown in Scheme 1, which exemplifies the preparation of racemic tofisopam. The racemic 2,3-benzodiazepine is prepared from the corresponding 2-benzopyrilium salt H by reaction with hydrazine hydrate, wherein X⁻ is a counterion such as, for example perchlorate:

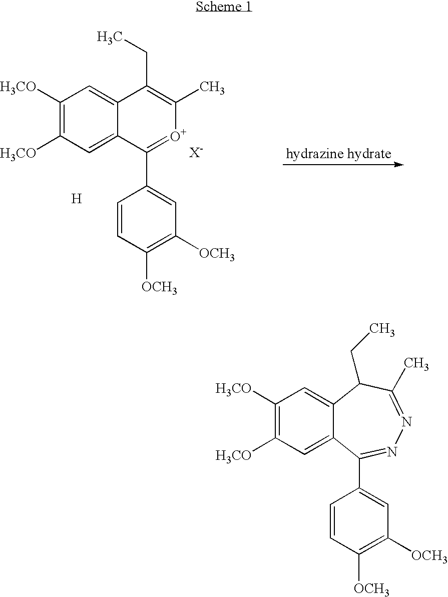

According to Scheme 1, hydrazine hydrate (98%, approximately 3 equivalents based on the 2-benzopyrylium salt) is added dropwise to a stirred solution of the 2-benzopyrylium salt H in glacial acetic acid (approximately 1 mL/3 mmol of 2-benzopyrylium salt). During this operation, the solution is maintained at an elevated temperature, preferably, 80-100° C. The solution is then maintained at an elevated temperature, preferably 95-100° C. for about one hour. Then the reaction mixture is diluted with 2% aqueous sodium hydroxide solution (approximately 3 equivalents based on the 2-benzopyrylium salt) and cooled. The product 2,3-benzodiazepine separates as a solid and is removed by filtration, washed with water and dried. The crude product may be purified by taking it up in a polar aprotic solvent such as dimethylformamide (DMF) at an elevated temperature, preferably 100-130° C. and decolorizing the solution with activated carbon. The carbon is removed by filtration and the filtered solution is diluted with water. The purified product precipitates out of the solution and is collected by filtration. See Kórósi et al., U.S. Pat. No. 4,322,346, the entire disclosure of which is incorporated herein by reference, disclosing three variations of the reaction protocol for preparing a substituted 2,3-benzodiazepine from the precursor benzopyrilium salt.

Retrosynthetically, the intermediate benzopyrilium salt, H, may be prepared from one of several starting materials. According to one such method, illustrated in Scheme 2, intermediate H is prepared from the corresponding aryl ethanol derivative D (3-(3,4-dimethoxyphenyl)pentan-2-ol) via the isochroman intermediate F (1-(3,4-dimethoxyphenyl)-4-ethyl-6,7-dimethoxy-3-methyliso-chromane) wherein X— is a counterion such as, for example perchlorate.

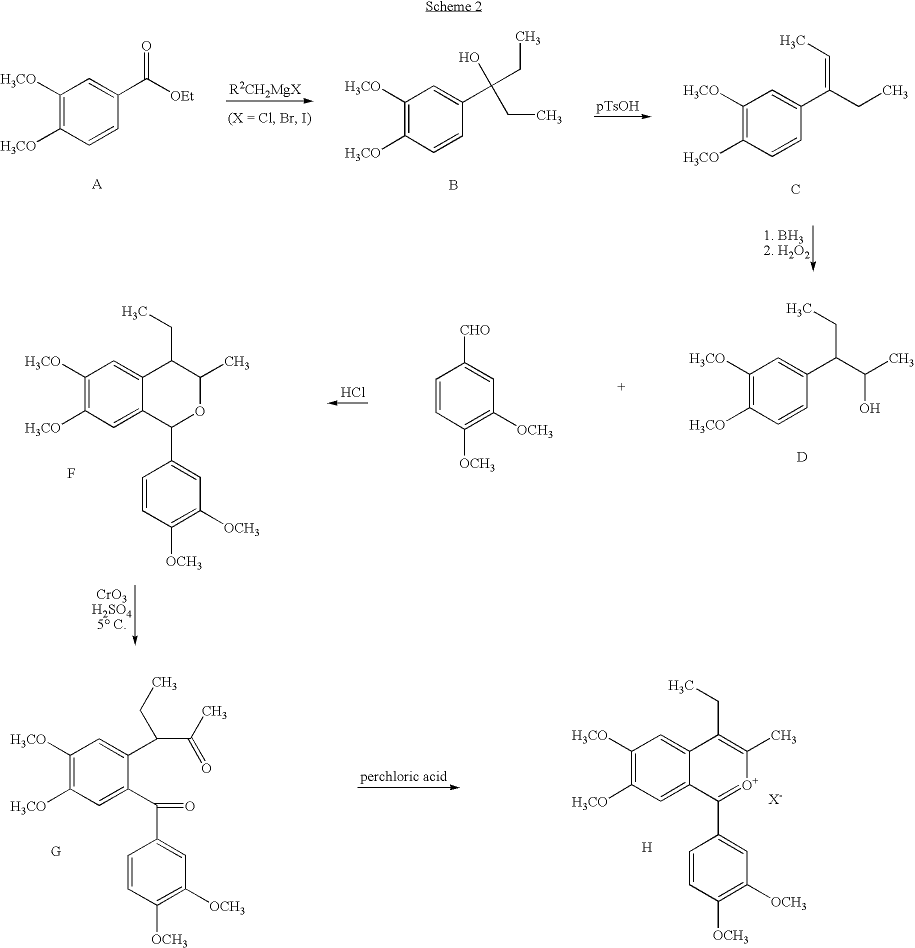

According to Scheme 2, ethyl-3,4-dimethoxybenzoate, A is dissolved in a suitable solvent, preferably ether and cooled to 0° C. Two equivalents of an ethyl Grignard reagent, such as ethyl magnesium iodide is added dropwise and the reaction is allowed to warm to room temperature and monitored for disappearance of starting material. When the reaction is complete, it may be quenched with a proton source such as acetic acid. Volatiles are removed in vacuo, and the product B (3-(3,4-dimethoxyphenyl)pentan-3-ol) is used for the next step without purification.

3-(3,4-Dimethoxyphenyl)pentan-3-ol), B, is taken up in a high boiling solvent such as toluene and a catalytic amount of para-toluene sulfonic acid (p-TsOH). The mixture is warmed to reflux and may be monitored for disappearance of starting materials. When the reaction is complete, the volatiles are removed in vacuo and the crude product C (4-((1Z)-1-ethylprop-1-enyl)-1,2-dimethoxybenzene) is purified by column chromatography.

4-((1Z)-1-Ethylprop-1-enyl)-1,2-dimethoxybenzene, C is hydroxylated under anti-Markovnikov conditions to give intermediate D (3-(3,4-dimethoxyphenyl)pentan-2-ol). A solution of D, and of 3,4-dimethoxybenzaldehyde, E (1.2 eq) are dissolved in anhydrous dioxane. The resulting solution is then saturated with gaseous HCl and warmed, preferably to reflux temperature for about one hour. The mixture is then cooled to room temperature, poured into water, basified, preferably with aqueous sodium hydroxide and extracted with an organic solvent, preferably ethyl acetate. The extract is dried, filtered and concentrated under vacuum. The resulting residue is purified, preferably by crystallization to yield F (1-(3,4-dimethoxyphenyl)-4-ethyl-6,7-dimethoxy-3-methylisochromane).

To a stirred, cooled, (preferably to 0-5° C.) solution of F (2 g) in acetone (30 mL), is added dropwise a solution of chromium trioxide (2 g) in 35% sulfuric acid (20 mL); added at a rate such that the reaction temperature remains below 5° C. After the addition is complete, the reaction mixture is allowed to rise to room temperature and is stirred at room temperature for two hours. The reaction mixture is then poured into water and extracted with an organic solvent, preferably ethyl acetate. The organic extract is washed with water and then with ice-cold 10% aqueous sodium hydroxide. The aqueous alkaline fraction is then acidified, preferably with dilute aqueous hydrochloric acid and extracted with an organic solvent, preferably, chloroform. The chloroform extract is dried, filtered and concentrated under vacuum to give G (3-{2-[(3,4-dimethoxyphenyl)carbonyl]-4,5-dimethoxyphenyl}pentan-2-one). The crude residue may further be purified by column chromatography.

G (5 g) is dissolved in glacial acetic acid (15 mL). To this mixture was added 60% perchloric acid (7.5 mL). The resulting mixture is warmed to 100° C. (steam bath) for three minutes. The mixture is allowed to cool to room temperature. Crystallization of the crude product may begin spontaneously at this point or may be induced by addition to the reaction mixture of ether or ethyl acetate. The product 2-benzopyrylium salt H is removed by filtration and purified by recrystallization, preferably from ethanol or glacial acetic acid/ethyl acetate.

A similar synthetic sequence for preparation of 2,3-benzodiazepines is disclosed in U.S. Pat. No. 3,736,315, the entire disclosure of which is incorporated herein by reference. Synthetic strategies for preparation of 2,3-benzodiazepines are disclosed in Horvath et al., Progress in Neurobiology 60(2000) pp. 309-342 and references cited therein; the entire disclosures of which are incorporated herein by reference. These synthetic sequences may be used to prepare racemic tofisopam.

Alternative methods for preparation of intermediate H start with an aryl acetonide or indanone starting material. See Kunnetsov, E. V., and Dorofeenko, G. N., Zh. Org. Khim., 6,578-581. and M. Vajda, Acta Chem. Acad. Sci. Hung., 40, 295-307, 1964, respectively.

Resolution of Racemic 2,3-Benzodiazepines

The synthetic procedures shown (or referenced) above produce racemic 2,3-benzodiazepines. In order to provide the (R)-2,3-benzodiazepines of Formula I useful in methods of the present invention, the racemic mixture must be resolved.

A racemic 2,3-benzodiazepine may be converted to the (S)-dibenzoyltartaric acid salt, which is a diastereomeric mixture of SS and RS configurations. The pair of diastereomers (R,S) and (S,S) possess different properties, e.g., differential solubilities, that allow for the use of conventional separation methods. Fractional crystallization of diastereomeric salts from a suitable solvent is one such separation method. This resolution has been successfully applied to the resolution of racemic tofisopam. See Hungarian Patent 178516 and also Toth et al., J Heterocyclic Chem., 20: 709-713 (1983), the entire disclosures of which are incorporated herein by reference.

Racemic 2,3-benzodiazepines may also be resolved without diastereomer formation by differential absorption on a chiral stationary phase of a chromatography column, particularly a preparative HPLC column. Chiral HPLC columns are commercially available with a variety of packing materials to suit a broad range of separation applications. Exemplary stationary phases suitable for resolving the racemic 2,3-benzodiazepines include:

(i) macrocyclic glycopeptides, such as silica-bonded vancomycin which contains 18 chiral centers surrounding three pockets or cavities;

(ii) chiral α₁-acid glycoprotein;

(iii) human serum albumin; and

(iv) cellobiohydrolase (CBH).

Chiral α₁-acid glycoprotein is a highly stable protein immobilized onto spherical silica particles that tolerates high concentrations of organic solvents, high and low pH, and high temperatures. Human serum albumin, though especially suited for the resolution of weak and strong acids, zwitterionic and nonprotolytic compounds, has been used to resolve basic compounds. CBH is a very stable enzyme that has been immobilized onto spherical silica particles and is preferentially used for the separation of enantiomers of basic drugs from many compound classes.

The resolution of tofisopam by chiral chromatography using macrocyclic glycopeptide as a stationary phase on a Chirobiotic V™ column (ASTEAC, Whippany, N.J.) is disclosed in U.S. Pat. No. 6,080,736. Fitos et al. (J. Chromatogr., 709: 265 (1995)), discloses another method for resolving racemic tofisopam by chiral chromatography using a chiral α₁-acid glycoprotein as a stationary phase on a CHIRAL-AGP™ column (ChromTech, Cheshire, UK). This method separates the (R)- and (S)-enantiomers and also resolves the two conformers (discussed below) of each enantiomer. The Chirobiotic V™ column is available in a semi-preparative size as employed for the above separation 500 mm×10 mm). In addition, the stationary phase of the Chirobiotic V™ column is commercially available in bulk for packing of preparative chromatography columns with larger sample capacity. The entire disclosures of the aforementioned patents and publications are incorporated herein by reference in their entireties. The disclosed methods may be utilized for resolving not only tofisopam, but also any other racemic 2,3-benzodiazepine of Formula I.

In addition to existing as (R)- and (S)-enantiomers, compounds of Formula I, exemplified by tofisopam may also exist in two stable conformations that may be assumed by the benzodiazepine ring as generally depicted below.

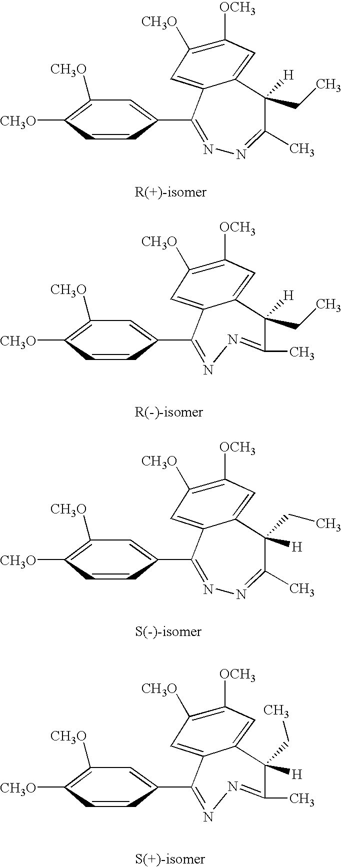

The present invention includes methods as described herein that use any and all observable conformations of compounds of Formula I.

Compounds of Formula I used in the practice of methods of the present invention may take the form of pharmaceutically-acceptable salts. The term “salts”, embraces salts commonly used to form alkali metal salts and to form addition salts of free acids or free bases. The term “pharmaceutically-acceptable salt” refers to salts that possess toxicity profiles within a range so as to have utility in pharmaceutical applications. Pharmaceutically unacceptable salts may nonetheless possess properties such as high crystallinity, which have utility in the practice of the present invention, such as for example utility in a synthetic process or in the process of resolving enantiomers from a racemic mixture.

Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, salicyclic, galactaric and galacturonic acid.

Suitable pharmaceutically acceptable base addition salts of compounds of Formula I, particularly compounds containing a group having a sufficiently acidic proton, e.g., an aromatic —OH group, may be prepared from a compound of Formula I with by reacting the Formula I compound with an appropriate base. Suitable base addition salts of compounds of Formula I include, for example, metallic salts made from calcium, magnesium, potassium, sodium and zinc or organic salts made from N,N′-dibenzylethylenediamine, chloroprocaine, choline, diethanolamine, ethylenediamine, meglumine (N-methylglucamine) and procaine.

All of the salts disclosed herein may be prepared by conventional means from a compound of Formula I, for example, by reacting the appropriate acid or base with a compound of Formula I.

The compounds useful in methods of the invention may be administered to individuals (mammals, including animals and humans) afflicted with disorders associated with elevated body temperature or with disorders wherein lowering the body temperature below the normal body temperature has therapeutic benefit.

For treating or preventing disorders associated with elevated body temperature or disorders wherein lowering the body temperature below the normal body temperature has therapeutic benefit, the specific dose of compound according to the invention to obtain therapeutic benefit will, of course, be determined by the particular circumstances of the individual patient including, the size, weight, age and sex of the patient. Also determinative will be the nature and stage of the disease and the route of administration. For example, a daily dosage of from about 100 to 1500 mg/kg/day may be utilized. Preferably, a daily dosage of from about 100 to 1000 mg/kg/day may be utilized. More preferably, a daily dosage of from about 100 to 500 mg/kg/day may be utilized. Higher or lower doses are also contemplated.

For prophylactic administration, a compound of Formula I should be administered far enough in advance of a known event that increases the body temperature, such that the compound is able to reach the site of action in sufficient concentration to exert a hypothermic effect. The pharmacokinetics of specific formulations may be determined by means known in the art and tissue levels of a compound of Formula I in a particular individual may be determined by conventional analyses.

The methods of the present invention may comprise administering one or more compounds of Formula I in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent or excipient that is compatible with the other ingredients of the formulation and not deleterious to the recipient.

One or more compounds useful in the practice of the present inventions may be administered simultaneously, by the same or different routes, or at different times during treatment or prevention therapy.

In addition, one or more compounds of Formula I may be administered to lower the body temperature of an individual suffering from hot flashes, particularly hot flashes associated with menopause, in combination with one or more additional therapeutic agents. Such additional agents include estrogen agonists, progesterone agonists, selective estrogen receptor modulators, bisphosphonates, SSRIs, NSRIs, and GABA modulators.

According to one embodiment, the one or more additional agents comprise an estrogen agonist and a progesterone agonist.

Estrogen agonists believed useful in combination with compounds of Formula I in methods of the invention include, for example, estradiol.

Progesterone agonists believed useful in combination with compounds of Formula I in methods of the invention include, for example, trimegestrone.

Selective estrogen receptor modulators believed useful in combination with compounds of Formula I in methods of the invention include, for example, raloxifene and bazedoxifene.

Bisphosphonates believed useful in combination with compounds of Formula I in methods of the invention include, for example, risedronic acid and ibandronic.

SSRIs believed useful in combination with compounds of Formula I in methods of the invention include, for example, fluoxetine and paroxetine.

NSRIs believed useful in combination with compounds of Formula I in methods of the invention include, for example, venlafaxine.

GABA modulators believed useful in combination with compounds of Formula I in methods of the invention include, for example, gabapentin.

The one or more additional therapeutic agents may be administered simultaneously with at least one Formula I compound, or may be administered separately. The compounds may be administered by the same or by different routes.

Where the at least one Formula I compound and the one or more additional therapeutic agents are administered at different times, the administration times are preferably optimized to obtain the therapeutic effect on hot flashes by the combination, based on the pharmacokinetic profiles of the compounds administered.

Where the at least one Formula I compound and the one or more additional therapeutic agents are administered simultaneously, the administration may be by the same or by different routes. Preferably, simultaneous administration is done by administering the compounds as part of the same pharmaceutical composition.

The active agent may be administered for therapeutic effect by any route, for example enteral (e.g., oral, rectal, intranasal, etc.) and parenteral administration. Parenteral administration includes, for example, intravenous, intramuscular, intraarterial, intraperitoneal, intravaginal, intravesical (e.g., into the bladder), intradermal, topical or subcutaneous administration. Also contemplated within the scope of the invention is the instillation of drug in the body of the patient in a controlled formulation, with systemic or local release of the drug to occur at a later time. For antiinflammatory use, the drug may be localized in a depot for controlled release to the circulation, or controlled release to a local site of inflammation.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th edition, (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agent. Stabilizing agents, antioxidizing agents and preservatives may also be added. Suitable antioxidizing agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The compositions of the present invention can also be formulated so as to provide slow or controlled-release of the active ingredient therein. In general, a controlled-release preparation is a composition capable of releasing the active ingredient at the required rate to maintain constant pharmacological activity for a desirable period of time. Such dosage forms can provide a supply of a drug to the body during a predetermined period of time and thus maintain drug levels in the therapeutic range for longer periods of time than other non-controlled formulations.

For example, U.S. Pat. No. 5,674,533 discloses controlled-release compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 discloses a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 discloses controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. The patents cited above are incorporated herein by reference.

Biodegradable microparticles can be used in the controlled-release formulations of this invention. For example, U.S. Pat. No. 5,354,566 discloses a controlled-release powder that contains the active ingredient. U.S. Pat. No. 5,733,566 describes the use of polymeric microparticles that release antiparasitic compositions. These patents are incorporated herein by reference.

The controlled-release of the active ingredient may be stimulated by various inducers, for example pH, temperature, enzymes, water, or other physiological conditions or compounds. Various mechanisms of drug release exist. For example, in one embodiment, the controlled-release component can swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels can be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The active agent is administered according to the present invention to patients suffering from conditions that manifest the symptom of hyperthermia, or elevated body temperature. Such conditions include for example, serotonin syndrome and malignant hyperthermia and perimenopause/menopause. In addition, the active agent is administered according to the present invention to patients suffering from conditions wherein lowering the body temperature to a level below normal body temperature provides therapeutic benefit. Such conditions include stroke and cerebral ischemia.

The practice of the invention is illustrated by the following non-limiting examples.

### EXAMPLES

### Example 1

### Preparation of (R)-Tofisopam

A. Synthesis of racemic tofisopam: 4.41 g (10 mmol) of 1-(3,4-dimethoxyphenyl)-3-methyl-4-ethyl-6,7-dimethoxyisobenzopyrilium chloride hydrochloride is dissolved in methanol (35 mL) at a temperature of 40° C. After cooling to 20-25° C., hydrazine hydrate (0.75 g, 15 mmol, dissolved in 5 mL methanol) is added. The reaction is monitored by HPLC and when complete, is evaporated to dryness. The residue is triturated with cold water (3 mL), filtered and dried to yield the crude (R,S)-1-(3,4-dimethoxyphenyl)-4-methyl-5-ethyl-7-hydroxy-8-methoxy-5H-2,3-benzo-diazepine which is subsequently triturated with hot ethyl acetate to yield the pure product.

B. Resolution of racemic tofisopam to produce (R)-tofisopam:

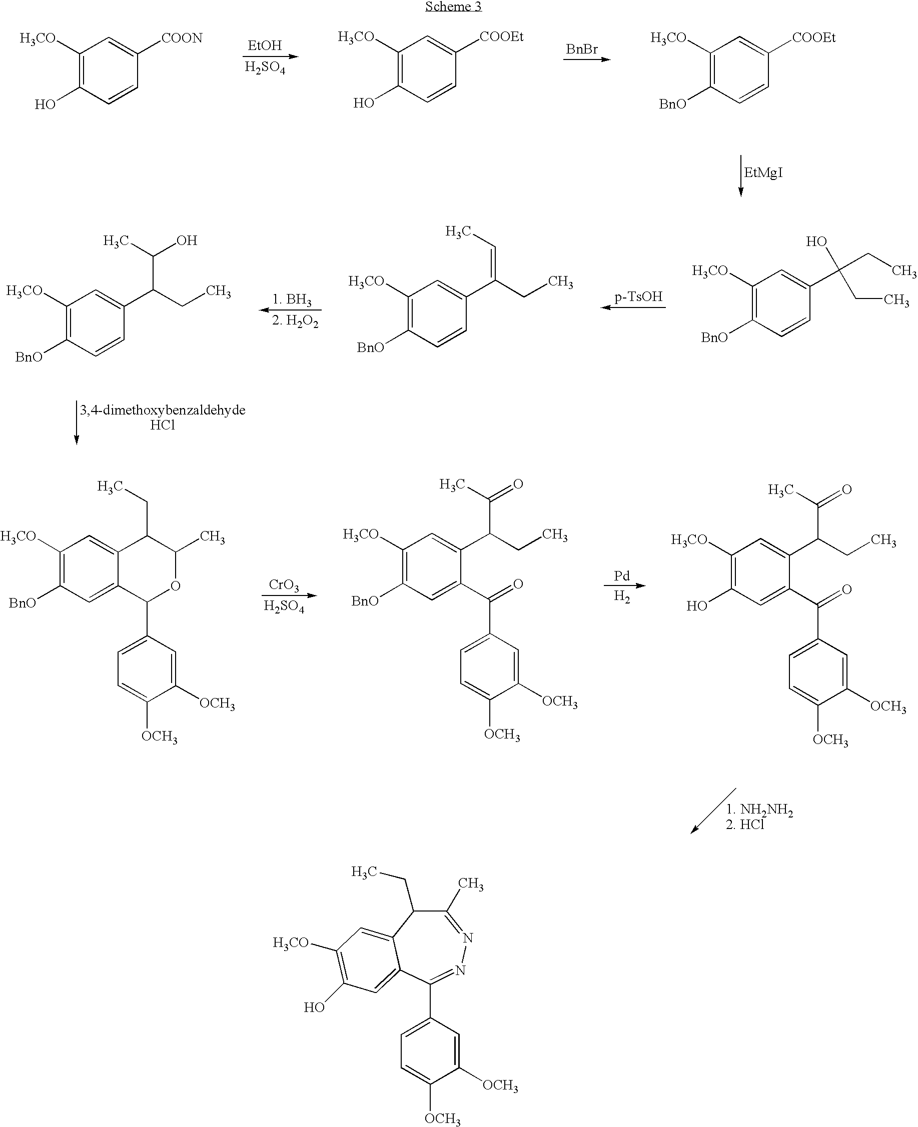

(i) Estenrfication of 3-methoxy-4-hydroxybenzoic acid to Yield ethyl-3-methoxy-4-hydroxybenzoate.

A solution of 100 g of 3-methoxy-4-hydroxybenzoic acid and 17 g of concentrated sulfuric acid in 300 mL of absolute ethanol was heated at reflux overnight. The mixture was concentrated and the residue poured into water. Methylene chloride was added and the solution washed successively with water, dilute sodium bicarbonate and water, then dried and concentrated. Yield: 118 g.

(ii) Benzylation of ethyl-3-methoxy-4-hydroxybenzoate to Yield ethyl-3-methoxy-4-benzyloxybenzoate.

A solution of 118 g of ethyl-3-methoxy-4-hydroxybenzoate and 86 mL of benzyl bromide in 600 mL of acetone containing a suspension of 124 g of potassium carbonate was heated at reflux overnight. The mixture was filtered, the filtrate concentrated and the residue recrystallized from acetone.

(iii) Addition of ethyl magnesium iodide to ethyl-3-methoxy-4-benzyloxybenzoate to Yield 3-(3-methoxy-4-benzyloxyphenyl)pentan-3-ol.

Iodoethane (112 mL) was added dropwise to a suspension of 35 g of magnesium turnings in 160 mL of ether. After the formation of ethyl magnesium iodide was complete, a solution of 142 g of ethyl 3-methoxy-4-benzyloxybenzoate in ether was added and the mixture was allowed to stir at room temperature for 3 days. The reaction was quenched by addition of saturated ammonium chloride. The layers were separated and the ether layer was dried and concentrated to an oily residue. Yield: 110 g.

(iv) Elimination of H₂O from 3-(3-methoxy-4-benzyloxyphenyl)pentan-3-ol to Yield 4-((1 Z)-1-ethylprop-1-enyl)-1-benzyloxy-2-methoxybenzene.

A solution of 110 g of crude 3-(3-methoxy-4-benzyloxyphenyl)pentan-3-ol and 7 g of p-tolenesulfonic acid in 2 L of benzene was heated at reflux for 4 hr with azeotropic removal of water. The mixture was then filtered through a pad of sodium bicarbonate and the filtrate concentrated. The residue was purified by column chromatography on neutral alumina.

(v) Addition of H₂O to 4-((1Z)-1-ethylprop-1-enyl)-1-benzyloxy-2-methoxybenzene to Yield 3-(3-methoxy-4-benzyloxyphenyl)pentan-2-ol.

To a solution of 96 g of 4-((1Z)-1-ethylprop-1-enyl)-1-benzyloxy-2-methoxybenzene in tetrahydrofuran at 0° C. was added 510 mL of a 1.0M solution of borane-tetrahydrofuran complex in tetrahydrofuran. The mixture was stirred for 3 hr at 0° C., then 204 mL of 25% hydrogen peroxide was added. The mixture was adjusted to pH 8 by addition of SM sodium hydroxide and extracted with ether. The combined ether extracts were dried and concentrated. Yield: 102 g.

(vi) Reaction of 3-(3-methoxy-4-benzyloxyphenyl)pentan-2-ol with 3,4-dimethoxybenzaldehyde to Yield 4-(4-ethyl-6-methoxy-7-benzyloxy-3-methyliso-chromanyl)-1,2-dimethoxybenzene.

A solution of 46 g of 3,4-dimethoxybenzaldehyde and 100 g of crude 3-(3-methoxy-4-benzyloxyphenyl)pentan-2-ol in 0.3 L of dioxane was saturated with hydrogen chloride gas. The mixture was heated at reflux for 3 hr, then poured into water, basified with dilute sodium hydroxide and extracted with methylene chloride. The combined methylene chloride extracts were dried and concentrated.

(vii) Ring-opening of 4-(4-ethyl-6-methoxy-7-benzyloxy-3-methyliso-chromanyl)-1,2-dimethoxybenzene to Yield 3-(4-benzyloxy-5-methoxy-2-{[3,4-dimethoxyphenyl]carbonyl}phenyl)pentan-2-one.

To a solution of 50 g of crude 4-(4-ethyl-6-methoxy-7-benzyloxy-3-methyliso-chromanyl)-1,2-dimethoxybenzene in acetone at 5° C. was added a solution of 50 g of chromic oxide in 500 mL of 35% sulfuric acid. The mixture was stirred at room temperature for 2 hr, neutralized by adding cold 10% sodium hydroxide and concentrated to remove acetone. Water was added and the mixture extracted with methylene chloride. The combined methylene chloride extracts were dried and concentrated. The residue was purified by column chromatography on silica gel. Yield: 18 g.

(viii) Debenzylation of 3-(4-benzyloxy-5-methoxy-2-{[3,4-dimethoxy-phenyl]carbonyl}phenyl)pentan-2-one to Yield 3-{2-[(3,4-dimethoxy-phenyl)carbonyl]-4-hydroxy-5-methoxyphenyl}pentan-2-one.

A solution of 18 g of 3-(4-benzyloxy-5-methoxy-2-{[3,4-dimethoxy-phenyl]carbonyl}phenyl)pentan-2-one in methylene chloride containing a suspension of 2 g of 10% palladium on carbon was hydrogenated at 80 psi for 1 hr. The mixture was filtered through diatomaceous earth and the filtrate concentrated. Yield: 15 g.

(ix) Annulation of 3-{2-[(3,4-dimethoxy-phenyl)carbonyl]-4-hydroxy-5-methoxyphenyl}pentan-2-One by Reaction with hydrazine to Yield 1-(3,4-dimethoxyphenyl)-4-methyl-5-ethyl-7-methoxy-8-hydroxy-5H-2,3-benzodiazepine.

A solution of 14 g of 3-{2-[(3,4-dimethoxy-phenyl)carbonyl]-4-hydroxy-5-methoxyphenyl}pentan-2-one and 4.7 mL of hydrazine in 280 mL of ethanol was heated at reflux for 0.5 hr. After allowing the solution to cool to room temperature, it was saturated with HCl gas. The mixture was then concentrated to a volume of about 5 mL, basified with concentrated ammonium hydroxide, and extracted with methylene chloride. The combined methylene chloride extracts were dried and concentrated, and the residue recrystallized from ethyl acetate/hexane. Yield: 1.5 g

The product 1-(3,4-dimethoxyphenyl)-4-methyl-5-ethyl-7-methoxy-8-hydroxy-5H-2,3-benzodiazepine was analyzed by HPLC, elemental analysis, GC/MS, proton NMR and differential scanning calorimetry (DSC). The data are as follows:

Purity: 98.36% by HPLC (% area). Column: Betasil Phenyl 4.6×150 mm. Mobile Phase: Acetonitrile:0.0.1M Phosphate Buffer (70:30). Flow Rate: 0.5 mL/min. Wavelength: 254 nm.

GC-MS; M/e=358; with the fragmentation pattern matching the proposed structure.

Differential scanning calorimetry (DSC): Temperature program 100° C. to 300° C. at 5° C./min, indicated molar purity =99.14% and melting point of 146.2° C.

Elemental analysis (calculated/analysis): % C-68.14/68.12; % H-6.63/6.63; N-7.43/7.20. The calculated values include 0.1M of residual ethyl acetate.

NMR (DCCl₃) (performed on GE QE 300): 1.08 ppm (t, 3H); 1.96 (s, 3H); 2.10 (m, 2H); 2.77 (m, 1H); 3.91 (s, 3H); 3.93 (s, 3H); 3.98 (s, 3H); 5.73 (bs, 1H); 6.70 (s, 1H); 6.80 (d, 1H); 6.95 (s, 1H); 7.00 (d, 1H); 7.58 (s, 1H).

B. Resolution of 1-(3,4-dimethoxyphenyl)-4-methyl-5-ethyl-7-methoxy-8-hydroxy-5H-2,3-benzodiazepine

The enantiomers of racemic-1-(3,4-dimethoxyphenyl)-4-methyl-5-ethyl-7-methoxy-8-hydroxy-5H-2,3-benzodiazepine are resolved by chiral chromatography as follows.

Racemic-1-(3,4-dimethoxyphenyl)-4-methyl-5-ethyl-7-methoxy-8-hydroxy-5H-2,3-benzodiazepine is loaded onto a semipreparative (500 mm×10 mm) Chirobiotic V column (ASTEC, Whippany, N.J.). Elution of the enantiomeric mixture with methyl-tert-butyl ether/acetonitrile (90/10 V/V), at a flow rate of 40 mL/min, is monitored at 310 nm. Fraction size is 10-20 mL and fractions are subjected to analytical chromatography using the same solvent composition on an analytical (150×4.6 mm) Chirobiotic V column. The fractions containing each isolated enatiomer are processed by removing the elution solvent in vacuo.

### Example 3

### Stress-Induced Hypothermia.

A. Introduction

Mice, individually housed overnight, were subjected to two sequential rectal temperature measurements ten minutes apart. The first measurement is the basal temperature (T₁), the second one the stress-enhanced temperature (T₂). The difference (delta T) is the stress-induced hyperthermia. See, Van der Heyden et al., “Stress-induced hyperthermia in singly housed mice,” Physiology and Behavior, 463-470, (1997).

B. Procedure

Test animals (group housed mice) were assigned to five groups of ten animals each. The test groups were dosed according to Table 2 below.

TABLE 2
Test animal groups for Stress Induced Hyperthermia assay.

| Group | Test substance | Dose (mg/kg) |
|---|---|---|
| 1 | Chlordiazepoxide | 5 |
| 2 | (R)-tofisopam | 64 |
| 3 | (racemic)-tofisopam | 64 |
| 4 | (S)-tofisopam | 64 |
| 5 | vehicle | — |

The test animals were isolated in an experimental room approximately one hour before lights off on the day before the test. On the day of testing, animals were taken quietly from the cage, held in a supine position, the rectal temperature was measured and the animal was placed back into the cage. The same procedure was repeated 10 minutes later. The first temperature (T₁), the second temperature (T₂) and the difference (delta T) were recorded. The test compounds were administered intraperitonealy 60 minutes before T₁, in order to prevent the stress of being injected from affecting the temperature measurements.

C. Results

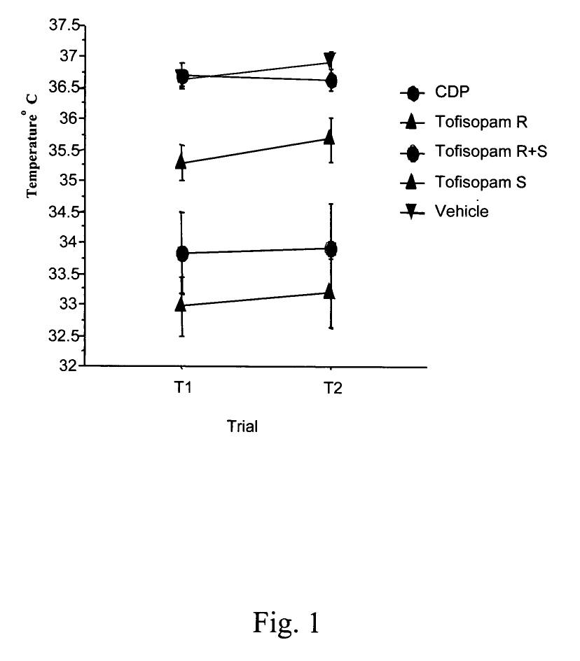

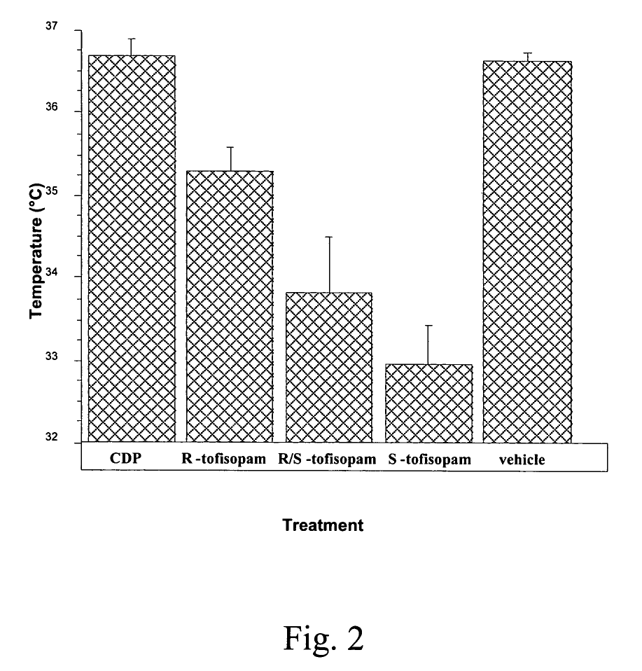

The core body temperatures T₁ and T₂ are shown in FIG. 1. The mean core body temperatures for T₁ are shown in FIG. 2. At both T₁ and T₂, racemic tofisopam demonstrates activity in lowering the core body temperature. The (S)-enantiomer of tofisopam is shown to be more active than either the racemate or the (R)-enantiomer. However, the (R)-enantiomer shows greater tolerability compared with either the racemate or the (S)-enantiomer. For example, the mice treated with the (R)-enantiomer show less sedation, abnormal gait, or ptosis, decreased muscle tone, decreased lacrimation, or decreased reactivity to touch compared with either (S)-enantiomer or the racemate.

All references cited herein are incorporated by reference. The present invention may be embodied in other specific forms without departing from the spirit or essential attributes thereof and, accordingly, reference should be made to the appended claims, rather than to the foregoing specification, as indication the scope of the invention.

The enantiomers of tofisopam were resolved by chiral chromatography. For example, tofisopam (42.8 mg dissolved in acetonitrile (ACN)) was loaded onto a Chirobiotic V column (ASTEC, Whippany, N.J.). Elution of the compounds with methyl-tert-butyl ether (MTBE)/ACN 90/10 (v/v), 40 mL/min, was monitored at 310 nm, 2 mm path. The R(+) enantiomer was the first compound to elute from the column. R(−) tofisopam (“peak A′”), S(−/+) tofisopam (“peak B” and “peak B′”), and residual R(+) tofisopam (“A”) co-eluted and were collected in a subsequent fraction.

The S(−) enantiomer was isolated from fraction 2 by the following protocol. Fraction 2 was dried, redissolved in 1 mL of ACN and loaded onto a Chirobiotic V column. Peak B and B′ was shave recycled over a Chirobiotic V column two more times (MTBE/ACN 90/10 (v/v), 40 mL/min monitored at 310 nm, 2 mm path). A peak containing S(−) tofisopam was collected from the third recycle, dried and stored for use in biological assays.

The final preparation of (S)-tofisopam was assayed for enantiomeric purity and found to be 87% pure (i.e., enantiomeric excess of 74%), as determined by analytical chromatography using Chiral Tech OD GH060 columns (Daicel) (hexane/IPA 90/10, 25° C., detection at 310 nm).

### EXAMPLE 2

### Preparation of (S)-1-(3,4-dimethoxyphenyl)-4-methyl-5-ethyl-7-methoxy-8-hydroxy-5H-2,3-benzodiazepine

A. Synthesis of racemic-1-(3,4-dimethoxyphenyl)-4-methyl-5-ethyl-7-methoxy-8-hydroxy-5H-2,3-benzodiazepine according to the route of Scheme 3.

What is claimed is:

1. A method of lowering body temperature of an individual, comprising administering to the individual an effective amount of at least one compound according to Formula I: 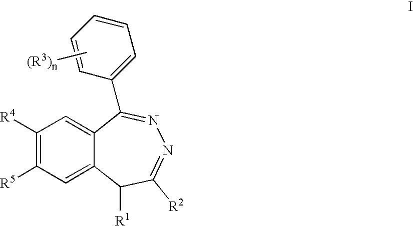
wherein:
R¹ is —(C₁-C₇)hydrocarbyl or —(C₂-C₆)heteroalkyl;
R² is selected from the group consisting of —H, and —(C₁-C₇)hydro-carbyl;
wherein R¹ and R² may combine to form a carbocyclic or heterocyclic 5- or 6-membered ring;
R^{3a}, R^{3b} and R^{3c} are independently selected from the group consisting of —H, —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁-C₆)alkyl, —NO₂, and halogen;
provided at least one of R^{3a}, R^{3b} and R^{3c} is other than —H;
R⁴ and R⁵ are independently selected from the group consisting of —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁ —C₆)alkyl, —NO₂, and halogen;
wherein R⁴ and R⁵ may combine to form a 5-, 6- or 7-membered heterocyclic ring; and
wherein the administered compounds according to Formula I comprise an (R)-enantiomer, substantially free of the corresponding (S)-enantiomer, with respect to the absolute conformation at the 5-position of the benzodiazepine ring; or
a pharmaceutically-acceptable salt of such a compound.

2. The method according to claim 1 wherein the administered compound is a compound according to Formula II: 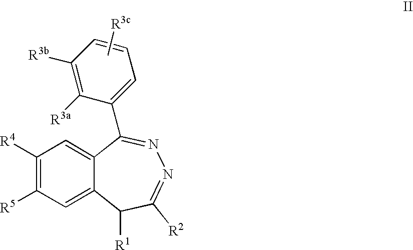
wherein:
R¹ is-(C₁-C₇)hydrocarbyl or —(C₂-C₆)heteroalkyl;
R² is selected from the group consisting of —H, and —(C₁-C₇)hydro-carbyl;
wherein R¹ and R² may combine to form a carbocyclic or heterocyclic 5- or 6-membered ring;
R^{3a}, R^{3b} and R^{3c} are independently selected from the group consisting of —H, —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁-C₆)alkyl, —NO₂, and halogen;
provided at least one of R^{3c} and R^{3b} is other than —H;
R⁴ and R⁵ are independently selected from the group consisting of —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁ —C₆)alkyl, —NO₂, and halogen;
wherein R⁴ and R⁵ may combine to form a 5-, 6- or 7-membered heterocyclic ring; and
wherein the administered compounds according to Formula I comprise an (R)-enantiomer, substantially free of the corresponding (S)-enantiomer, with respect to the absolute conformation at the 5-position of the benzodiazepine ring; or
a pharmaceutically-acceptable salt of such a compound.

3. The method according to claim 2;
wherein:
R^{3c} is —H;
one or two of R^{3a}, R^{3b}, R⁴, and R⁵ is —OH; and
the remaining members of the group R^{3a}, R^{3b}, R⁴, R¹ are independently selected from the group consisting of —O(C₁-C₇)hydrocarbyl, —OC(═O)(C₁-C₆)alkyl, —OH, —SH, —S(C₁-C₃)alkyl, —NH₂, —NH(C₁-C₆)alkyl, —N((C₁-C₆)alkyl)₂, —NH(═O)(C₁-C₆)alkyl, —NO₂, and halogen;
wherein R⁴ and R⁵ may combine to form a 5-, 6- or 7-membered heterocyclic ring.

4. The method according to claim 3, wherein:
one or two of R^{3a}, R^{3b}, R⁴, and R⁵ is —OH;
one of the remaining members of the group R^{3a}, R^{3b}, R⁴, and R⁵ is —O(C₁-C₇)hydrocarbyl; and
the remaining members of the group R^{3a}, R^{3b}, R⁴, and R⁵ are independently selected from the group consisting of —O(C₁-C₇)hydrocarbyl, —OC(═O)(C₁ —C₆)alkyl, —SH, —S(C₁-C₃)alkyl, —NH₂, —NH(C₁-C₆)alkyl, —N((C₁-C₆)alkyl)₂, —NH(═O)(C₁-C₆)alkyl, —NO₂ and halogen;
wherein R⁴ and R⁵ may combine to form a 5-, 6- or 7-membered heterocyclic ring.

5. The method according to claim 4, wherein:
one or two of R^{3a}, R b, R⁴, and R⁵ is —OH; and
the remaining members of the group R^{3a}, R^{3b}, R⁴, and R⁵ are independently selected from the group consisting of —O(C₁-C₇)hydrocarbyl.

6. The method according to claim 5, wherein:
the compound according to Formula II is selected from the group consisting of:
(R)-1-(3,4-dimethoxyphenyl)-4-methyl-5-ethyl-7-hydroxy-8-methoxy-5H-2,3-benzodiazepine;
(R)-1-(3-hydroxy-4-methoxyphenyl)-4-methyl-5-ethyl-7,8-dimethoxy-5H-2,3-benzodiazepine;
(R)-1-(3-methoxy-4-hydroxyphenyl)-4-methyl-5-ethyl-7,8-dimethoxy-5H-2,3-benzodiazepine;
(R)-1-(3,4-dimethoxyphenyl)-4-methyl-5-ethyl-7-methoxy-8-hydroxy-5H-2,3-benzodiazepine;
(R)-1-(3-methoxy-4-hydroxyphenyl)-4-methyl-5-ethyl-7-hydroxy-8-methoxy-5H-2,3-benzodiazepine;
(R)-1-(3-hydroxy-4-methoxyphenyl)-4-methyl-5-ethyl-7-hydroxy-8-methoxy-5H-2,3-benzodiazepine; and
pharmaceutically-acceptable salts of such compounds.

7. The method according to claim 2 wherein:
R^{3c} is —H; and
R^{3a}, R^{3b}, R⁴, and R⁵ are independently selected from the group consisting of —(C₁-C₇)hydrocarbyl.

8. The method according to claim 7, wherein the administered compound is (R)-tofisopam, or a pharmaceutically-acceptable salt thereof.

9. The method according to claim 1 wherein said individual is afflicted with a disorder associated with an elevated body temperature.

10. The method according to claim 9 wherein the disorder is fever.

11. The method according to claim 9 wherein the disorder is malignant hyperthermia.

12. The method according to claim 9 wherein the disorder is serotonin syndrome.

13. The method according to claim 9 wherein the disorder comprises hot flashes.

14. The method according to claim 13 wherein said hot flashes occur during menopause.

15. The method according to claim 13 wherein said hot flashes occur during perimenopause.

16. The method of claim 13 wherein said hot flashes are side effects of drug therapy.

17. The method of claim 13 wherein said hot flashes occur subsequent to the removal of estrogen-producing tissue.

18. The method of claim 1 wherein said individual is afflicted with a disorder wherein therapeutic benefit is achieved by lowering of the body temperature to a level below the normal body temperature.

19. The method of claim 18 wherein the disorder is cerebral ischemia.

20. The method of claim 16 wherein the disorder is stroke.

21. A method of lowering body temperature of an individual suffering from hot flashes associated with menopause, comprising administering to said individual an effective amount of
(a) at least one compound according to Formula I: 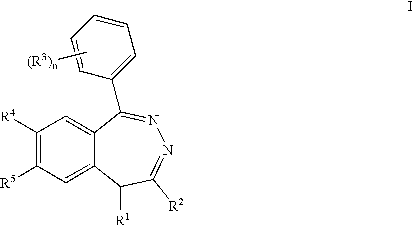
wherein:
R¹ is —(C₁-C₇)hydrocarbyl or —(C₂-C₆)heteroalkyl;
R² is selected from the group consisting of —H, and —(C₁-C₇)hydro-carbyl;
wherein R¹ and R² may combine to form a carbocyclic or heterocyclic 5- or 6-membered ring;
R^{3a}, R^{3b} and R^{3c} are independently selected from the group consisting of —H, —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁-C₆)alkyl, —NO₂, and halogen;
provided at least one of R^{3a}, R^{3b} and R^{3c} is other than —H;
R⁴ and R₅ are independently selected from the group consisting of —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁ —C₆)alkyl, —NO₂, and halogen;
wherein R⁴ and R⁵ may combine to form a 5-, 6- or 7-membered heterocyclic ring; and
wherein the administered compounds according to Formula I comprise an (R)-enantiomer, substantially free of the corresponding (S)-enantiomer, with respect to the absolute conformation at the 5-position of the benzodiazepine ring; or
a pharmaceutically-acceptable salt of such a compound; and
(b) one or more additional therapeutic agents selected from the group consisting of estrogen agonists, progesterone agonists, selective estrogen receptor modulators, bisphosphonates, selective serotonin reuptake inhibitors, norepinephrine serotonin reuptake inhibitors and gamma aminobuteric acid modulators.

22. The method according to claim 21, wherein the one or more additional therapeutic agents comprises an estrogen agonist and a progesterone agonist.

23. The method according to claim 21 or claim 22, wherein the estrogen agonist is estradiol.

24. The method according to claim 21 or 22, wherein the progesterone agonist is trimegestrone.

25. The method according to claim 21, wherein the selective estrogen receptor modulator agonist is selected from the group consisting of raloxifene and bazedoxifene.

26. The method according to claim 21, wherein the bisphosphonate is selected from the group consisting of risedronic acid and ibandronic.

27. The method according to claim 21, wherein the selective serotonin reuptake inhibitor is selected from the group consisting of fluoxetine and paroxetine.

28. The method according to claim 21, wherein the norepinephrine serotonin reuptake inhibitor is venlafaxine.

29. The method according to claim 21, wherein the GABA modulator is gabapentin.

30. A composition comprising
(a) at least one compound of Formula I: 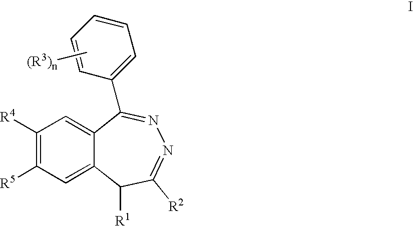
wherein:
R¹ is —(C₁-C₇)hydrocarbyl or —(C₂-C₆)heteroalkyl;
R² is selected from the group consisting of —H, and —(C₁-C₇)hydro-carbyl;
wherein R¹ and R² may combine to form a carbocyclic or heterocyclic 5- or 6-membered ring;
R^{3a}, R^{3b} and R^{3c} are independently selected from the group consisting of —H, —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁-C₆)alkyl, —NO₂, and halogen;
provided at least one of R^{3a}, R^{3b} and R^{3c} is other than —H;
R⁴ and R⁵ are independently selected from the group consisting of —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁-C₆)alkyl, —NO₂, and halogen;
wherein R⁴ and R⁵ may combine to form a 5-, 6- or 7-membered heterocyclic ring; and
wherein the administered compounds according to Formula I comprise an (R)-enantiomer, substantially free of the corresponding (S)-enantiomer, with respect to the absolute conformation at the 5-position of the benzodiazepine ring; or
a pharmaceutically-acceptable salt of such a compound; and
(b) at least one additional therapeutic agent selected from the group consisting of estrogen agonists, progesterone agonists, selective estrogen receptor modulators, bisphosphonates; selective serotonin reuptake inhibitors, norepinephrine serotonin reuptake inhibitors and gamma aminobuteric acid modulators.

31. A method of treating an individual suffering from the symptoms associated with menopause, comprising administering to said individual an effective amount of at least one compound according to Formula I: 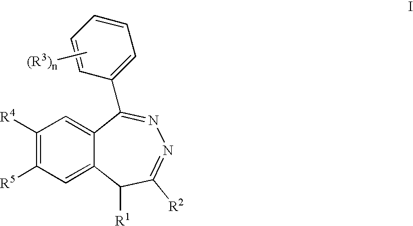
wherein:
R¹ is —(C₁-C₇)hydrocarbyl or —(C₂-C₆)heteroalkyl;
R² is selected from the group consisting of —H, and —(C₁-C₇)hydro-carbyl;
wherein R¹ and R² may combine to form a carbocyclic or heterocyclic 5- or 6-membered ring;
R^{3a}, R^{3b} and R^{3c} are independently selected from the group consisting of —H, —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁-C₆)alkyl, —NO₂, and halogen;
provided at least one of R^{3a}, R^{3b} and R^{3c} is other than —H;
R⁴ and R₅ are independently selected from the group consisting of —O(C₁-C₇)hydrocarbyl, —OH, —OC(═O)(C₁-C₆)alkyl, —OC(═O)O(C₁-C₇)hydrocarbyl, —SH, —S(C₁-C₃)alkyl, —NH₂, —NH(C₁-C₆)alkyl, —N((C₁-C₆)alkyl)₂, —NH(═O)(C₁ —C₆)alkyl, —NO₂, and halogen;
wherein R⁴ and R⁵ may combine to form a 5-, 6- or 7-membered heterocyclic ring; and
wherein the administered compounds according to Formula I comprise an (R)-enantiomer, substantially free of the corresponding (S)-enantiomer, with respect to the absolute conformation at the 5-position of the benzodiazepine ring; or a pharmaceutically-acceptable salt of such a compound.
